(19) United States
(12) Patent Application Publication
(10) Pub. No.: US 2005/0004130 A1
(43) Pub. Date: Jan. 6, 2005

(54) New metabotropic glutamate receptor compounds

(75) Inventors: Helena Gyback, Mariefred (SE); Martin Johansson, Sodertalje (SE); Alexander Minidis, Huddinge (SE); Gunnar Nordvall, Ronninge (SE); Patrick Raboisson, Huddinge (SE); David Wensbo, Grodinge (SE)

Correspondence Address: FOLEY AND LARDNER;SUITE 500, 3000 K STREET NW, WASHINGTON, DC, 20007, US

(73) Assignee: AstraZeneca and NPS Pharmaceuticals, Inc.

(21) Appl. No.: 10/766,948

(22) Filed: Jan. 30, 2004

Related U.S. Application Data
(60) Provisional application No. 60/443,889, filed on Jan. 31, 2003.
(60) Provisional application No. 60/530,632, filed on Dec. 19, 2003.

(51) Int. Cl. A61K 31/498; C07D 487/02

(52) U.S. Cl. 514/249; 544/350

(57) ABSTRACT

The present invention relates to new compounds of formula I, 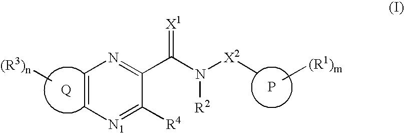
wherein P, Q, X¹, X², R¹, R², R³, R⁴, m and n, are as defined as in formula I, or N₁-oxides, salts, solvates or solvated salts thereof, processes for their preparation and new intermediates used in the preparation thereof, pharmaceutical compositions containing said compounds and to the use of said compounds in therapy.

### FIELD OF THE INVENTION

The present invention relates to a new class of compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Eight distinct mGluR subtypes, termed mGluRI through mGluR₈, have been identified by molecular cloning. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR¹, mGluR5 and their alternatively spliced variants. The binding of agonists to these receptors results in the activation of phospholipase C and the subsequent mobilization of intracellular calcium.

Neurological, Psychiatric and Pain Disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1(1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated. Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors have been suggested to play roles in a variety of acute and chronic pathophysiological processes and disorders affecting the CNS. These include stroke, head trauma, anoxic and ischemic injuries, hypoglycemia, epilepsy, neurodegenerative disorders such as Alzheimer's disease, psychiatric disorders and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial in all conditions underlain by excessive glutamate-induced excitation of CNS neurons, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K., Holloway, R. H., Penagini, R., Blackshaw, L. A., Dent, J., 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A.J.P.M, 2000; Diagnosis of reflux disease. Baillière's Clin. Gastroenterol. 14, pp. 759-774.

Because of their physiological and pathophysiological significance, there is a need for new potent mGluR agonists and antagonists that display a high selectivity for mGluR subtypes, particularly the Group I receptor subtype.

Prior Art

In pharmaceutical industry it is preferred to develop compounds that are easily absorbed after administration. Generally, improved solubility of a compound will improve the absorption of the compound after administration.

WO 99/26927 describes, among others, quinoxaline compounds that exhibit an inhibitory effect on the mGluR Group I receptors.

The object of the present invention is to provide compounds exhibiting an activity at metabotropic glutamate receptors (mGluRs), especially at the Group I receptor subtype, having improved solubility compared to the compounds described in WO 99/26927.

This is, among others, achieved through saturation of ring Q in the compounds of formula I.

### SUMMARY OF THE INVENTION

The present invention provides a compound of formula I 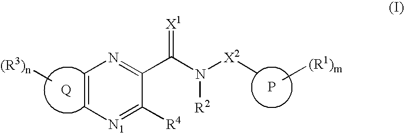
wherein:
X¹ is O or S;
X² is a bond or C₁₋₃alkyl;
P is C₃₋₇cycloalkyl or C₄₋₇cycloalkenyl;
R¹ is hydrogen, C₁₋₆alkyl, cyano, halogen and C₁₋₆alkylhalo, and one or more R¹ may be connected to each other or to one of the atoms that constitutes P to form a bridge or spirocyclo;

R² is hydrogen, C₁₋₃alkyl, fluoromethyl, difluoromethyl, trifluoromethyl, methoxy, fluoromethoxy, difluoromethoxy, trifluoromethoxy, C₀₋₃alkylamino, C₀₋₃alkylhydroxy or C₀₋₃alkyldimethylamino;
R⁴ is hydrogen, C₁₋₃alkyl, fluoromethyl, difluoromethyl, trifluoromethyl, methoxy, fluoromethoxy, difluoromethoxy, trifluoromethoxy, C₀₋₃alkylamino, C₀₋₃alkylhydroxy or C₀₋₃alkyldimethylamino;
Q is a ring containing 4, 5, 6 or 7 atoms independently selected from C, S, O and N, which may be saturated or partially unsaturated and said ring may further contain groups independently selected from SO, SO_{2,} CO, cyano and CS;
R³ is hydrogen, hydroxy, halogen, nitro, cyano, OC₁₋₃alkylhalo, C₁₋₃alkylhalo, C₁₋₃alkyl, C₁₋₃alkoxyC₀₋₃alkyl, C₀₋₃alkylOC₂₋₄alkanol C₁₋₃alkanol, amino, C₁₋₃alkylaminoC₀₋₃alkyl, (C₁₋₃alkyl)₂aminoC₀₋₃alkyl, amide, C₁₋₃alkylamideC₀₋₃alkyl or (C₁₋₃alkyl)₂amideC₀₋₃alkyl;
n is 0, 1, 2, 3 or 4; and
m is 0, 1, 2, 3 or 4;
or N₁-oxides, salts, solvates or solvated salts thereof.

In a further aspect of the invention there is provided pharmaceutical compositions comprising a therapeutically effective amount of the compound of formula I and a pharmaceutically acceptable diluent, excipients and/or inert carrier.

In yet a further aspect of the invention there is provided a pharmaceutical composition comprising the compound of formula I for use in the treatment of Group I mGluR receptor mediated disorders, and for use in the treatment of neurological disorders, psychiatric disorders, gastrointestinal disorders and pain disorders.

In still a further aspect of the invention there is provided the compound of formula I for use in therapy, especially for the treatment of Group I mGluR receptor mediated disorders, and for the treatment of neurological disorders, psychiatric disorders, gastrointestinal disorders and pain disorders.

In another aspect of the invention there is provided a processes for the preparation of compounds of formula I, and the intermediates used in the preparation thereof.

These and other aspects of the present invention are described in greater detail herein below.

### DETAILED DESCRIPTION OF THE INVENTION

The object of the present invention is to provide compounds exhibiting an activity at metabotropic glutamate receptors (mGluRs), especially at the group I receptors, as well as having a good absorbtion.

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms.

In the case where a subscript is the integer 0 (zero) the group to which the subscript refers to indicates that the group is absent.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term C₁₋₃alkyl has 1 to 3 carbon atoms and may be methyl, ethyl, n-propyl or i-propyl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

In this specification, unless stated otherwise, the term “cycloalkenyl” refers to an optionally substituted, non-aromatic cyclic hydrocarbon ring system containing one or two double-bonds. The term “C₄₋₇cycloalkenyl” may be, but is not limited to cyclobutenyl, cyclopentenyl, cyclohexenyl or cycloheptenyl and a cyclopentenyl group may for example be cyclopenten-3-yl or cyclopenten-4-yl,

In this specification, unless stated otherwise, the term “alkoxy” includes both straight or branched alkoxy groups. C₁₋₃alkoxy may be, but is not limited to methoxy, ethoxy, n-propoxy or i-propoxy.

In this specification, unless stated otherwise, the term “alkanol” includes both straight and branched chain alkanol groups. The term C₁₋₃alkanol having 1 to 3 carbon atoms and one hydroxy group may be, but is not limited to methanol, ethanol or propanol and a propanol group may for example be 1-propanol or 2-propanol.

In this specification, unless stated otherwise, the term “ring containing 4, 5, 6 or 7 atoms independently selected from C, S, O and N, which may be saturated or partially unsaturated and said ring may further contain groups independently selected from SO, SO₂, CO, C═N and CS” includes non-aromatic carbocyclic and heterocyclic rings.

Examples of such rings may be, but are not limited to cyclohexyl, cyclohexenyl, cyclopentyl, cyclopentenyl, imidazolidinyl, imidazolinyl, morpholinyl, piperazinyl, piperidyl, piperidonyl, pyrazolidinyl, pyrazolinyl, pyrrolidinyl, pyrrolinyl, tetrahydropyranyl or thiomorpholinyl. For example, in the structure below, Q is defined as cyclohexyl.

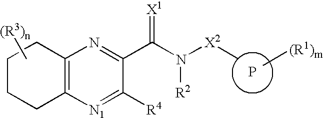

In this specification, unless stated otherwise, the term “bond” may be a saturated or unsaturated bond.

In this specification, unless stated otherwise, the term “halo” and “halogen” may be fluoro, chloro or bromo.

In this specification, unless stated otherwise, the term “alkylhalo” means an alkyl group as defined above, which is substituted with halo as described above.

The term “C₁₋₆alkylhalo” may include, but is not limited to fluoromethyl, difluoromethyl, trifluoromethyl, fluoroethyl, difluoroethyl or bromopropyl.

The term “OC₁₋₆alkylhalo” may include, but is not limited to fluoromethoxy, difluoromethoxy, trifluoromethoxy, fluoroethoxy or difluoroethoxy.

In this specification, unless stated otherwise, the term “bridge” means a molecular fragment, containing one or more atoms, or a bond, which connects two remote atoms in a ring, thus forming either bi- or tricyclic systems.

In this specification, unless stated otherwise, the term “spirocycle” defines a molecule or fragment in which two rings are connected to each other via one single atom that simultaniously constitutes one of the atoms in each ring.

In one embodiment of the invention P is C₃₋₇cycloalkyl or C₄₋₇cycloalkenyl. In another embodiment of the invention P is a C₅₋₇cycloalkyl. In a further embodiment of the invention P is selected from the group consisting of cyclopentane, cyclohexane and cycloheptane. In yet another embodiment P is cyclohexane.

In yet another embodiment of the invention P is C₄₋₇cycloalkenyl. In yet a further embodiment P is selected from the group consisting of cyclopentenyl, cyclohexenyl and cycloheptenyl.

In one embodiment of the invention P is substituted with 0, 1, 2, 3 or 4 groups R¹, wherein the number of R¹ substituents on the P ring is designated by the term m. In another embodiment of the invention m is 1 or 2.

In a further embodiment of the invention ring P is substituted by R¹ on position 2, 3 and/or 4 counting from the attachement-point of X² at position 1. In yet another embodiment ring P is substituted by one or two R¹ on position 4.

In yet a further embodiment of the invention the P ring is cyclohexyl and substituted at position 4 with one or two methyl groups.

In one embodiment of the invention R¹ is selected from the group consisting of hydrogen, C₁₋₆alkyl, cyano, halogen and C₁₋₆alkylhalo.

In another embodiment R¹ is hydrogen, C₁₋₆alkyl and one or more R¹ may be connected to each other or to one of the atoms that constitutes P to form a bridge or spirocyclo.

In a further embodiment of the invention R¹ is C₁₋₆alkyl. In yet another embodiment R¹ is methyl.

The present invention relates to the compound of formula I, wherein P is C₃₋₇cycloalkyl substituted with one or more R¹, wherein R¹ is hydrogen, C₁₋₆alkyl, cyano, halogen or C₁₋₆alkylhalo, and one or more R¹ may be connected to each other or to one of the atoms that constitutes P to form a bridge or spirocyclo.

The present invention also relates to the compound of formula I, wherein P is C₅₋₇cycloalkyl substituted with one or more R¹, wherein R¹ is methyl.

The present invention further relates to the compound of formula I, wherein P is C₄₋₇cycloalkenyl substituted with one or more R¹, wherein R¹ is C₁₋₆alkyl, cyano, halogen or C₁₋₆alkylhalo, and one or more R¹ may be connected to each other or to one of the atoms that constitutes P to form a bridge or spirocyclo.

In one embodiment of the invention X¹ is oxygen or sulfur. The present invention relates to the compound of formula I, wherein X¹ is oxygen.

In another embodiment of the invention the P ring is connected to the nitrogen by X², wherein X² may be a bond or a linker group C₁₋₃alkyl. The present invention further relates to the compound of formula I, wherein and X² is a bond.

In a further embodiment of the invention R² is selected from the group consisting of hydrogen, C₁₋₃alkyl, fluoromethyl, difluoromethyl, trifluoromethyl, methoxy, fluoromethoxy, difluoromethoxy, trifluoromethoxy, C₀₋₃alkylamino, C₀₋₃alkylhydroxy and C₀₋₃alkyldimethylamino. Yet another embodiment of the invention relates to the compound of formula I, wherein R² is hydrogen or methyl. The present invention also relates to the compound of formula I, wherein R² is hydrogen.

5 In yet a further embodiment of the invention R⁴ is selected from the group consisting of hydrogen, C₁₋₃alkyl, fluoromethyl, difluoromethyl, trifluoromethyl, methoxy, fluoromethoxy, difluoromethoxy, trifluoromethoxy, C₀₋₃alkylamino, C₀₋₃alkylhydroxy and C₀₋₃alkyldimethylamino. The present invention also relates to the compound of formula I, wherein R⁴ is hydrogen or methyl.

The present invention further relates to the compound of formula I, wherein R² is hydrogen and R⁴ is methyl.

In one embodiment of the invention Q is a ring containing 4, 5, 6 or 7 atoms independently selected from C, S, O and N, which may be saturated or partially unsaturated and said ring may further contain groups independently selected from SO, SO₂, CO, and CS.

The invention relates to the compound of formula I, wherein Q is a ring containing 5, 6 or 7 atoms independently selected from C, O and N, which may be saturated or partially unsaturated.

In another embodiment Q is a saturated C₅₋₇cycloalkyl ring. In a further embodiment Q is cyclopentane, cyclohexane or cycloheptane.

Q may be substituted with 0, 1, 2, 3 or 4 groups R³, wherein the number of R³ substituents on the Q ring is designated by the term n. In a further embodiment of the invention n is 0, 1, 2 or 3.

In yet another embodiment of the invention R³ is selected from the group consisting of hydrogen, hydroxy, halogen, nitro, cyano, OC₁₋₃alkylhalo, C₁₋₃alkylhalo, C₁₋₃alkyl, C₁₋₃alkoxyC₀₋₃alkyl, C₀₋₃alkylOC₂₋₄alkanol, C₁₋₃alkanol, amino, C₁₋₃alkylaminoC₀₋₃alkyl, (C₁₋₃alkyl)₂aminoC₀₋₃alkyl, amide, C₁₋₃alkylamideC₀₋₃alkyl and (C₁₋₃alkyl)₂amideC₀₋₃alkyl.

In yet a further embodiment of the invention R³ is hydrogen, hydroxy, halogen, cyano, C₁₋₃alkyl or C₁₋₃alkoxyC₀₋₃alkyl.

The present invention relates to the compound of formula I, wherein R³ is hydrogen, hydroxy, halogen, cyano, C₁₋₃alkyl or C₁₋₃alkoxyC₀₋₃alkyl.

In one embodiment of the invention R³ is hydrogen, hydroxy, fluor, cyano, fluoromethyl, methyl, methoxy, methanol, amino or carboxamide.

In another embodiment of the invention R³ is hydroxy or methyl.

The present invention also relates to the compound of formula I, wherein Q is a saturated C₅₋₇cycloalkyl ring substituted with one or more R³, wherein R³ is hydrogen, hydroxy, halogen, nitro, OC₁₋₃alkylhalo, C₁₋₃alkylhalo, C₁₋₃alkyl, C₁₋₃alkoxyC₀₋₃alkyl, C₁₋₃alkanol, cyano, amino or carboxamide.

The present invention relates to compounds of formula I as hereinbefore defined as well as to the N₁-oxides, salts, solvates or solvated salts thereof.

The invention is also related to the following compounds;
N-(trans-4-methylcyclohexyl)-5,6,7,8-tetrahydroquinoxaline-2-carboxamide,
N-(4,4-dimethylcyclohexyl)-5,6,7,8-tetrahydroquinoxaline-2-carboxamide,
N-(4,4-dimethylcyclohexyl)-3-methyl-5,6,7,8-tetrahydroquinoxaline-2-carboxamide,
8-methyl-N-(trans-4-methylcyclohexyl)-5,6,7,8-tetrahydroquinoxaline-2-carboxamide,
7-hydroxy-5,7-dimethyl-N-(trans-4-methylcyclohexyl)-6,7-dihydro-5H-cyclopenta[b]pyrazine-2-carboxamide,
N-(trans-4-methylcyclohexyl)-6,7,8,9-tetrahydro-5H-cyclohepta[b]pyrazine-2-carboxamide,
7-methyl-N-(trans-4-methylcyclohexyl)-5,6,7,8-tetrahydroquinoxaline-2-carboxamide,
6-methyl-N-(trans-4-methylcyclohexyl)-5,6,7,8-tetrahydroquinoxaline-2-carboxamide,
N-(trans-4-methylcyclohexyl)-6,7-dihydro-5H-cyclopenta[b]pyrazine-2-carboxamide,
N-(trans-4-methylcyclohexyl)-7,8-dihydro-5H-pyrano[3,4-b]pyrazine-2-carboxamide,
N-(trans-4-methylcyclohexyl)-7,8-dihydro-5H-pyrano[3,4-b]pyrazine-3-carboxamide,
7-hydroxy-N-(trans-4-methylcyclohexyl)-5,6,7,8-tetrahydroquinoxaline-2-carboxamide,
6-hydroxy-N-(trans-4-methylcyclohexyl)-5,6,7,8-tetrahydroquinoxaline-2-carboxamide,
N-(4,4-dimethylcyclohexyl)-5,6,7,8-tetrahydroquinoxaline-2-carboxamide 4-oxide and 6,7-dimethyl-N-(4-methylcyclohexyl)-6,7-dihydro-5H-cyclopenta[b]pyrazine-2-carboxamide,
or salts, solvates or solvated salts thereof.

One embodiment of the invention relates to compounds for formula I 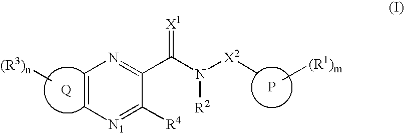
wherein:
X¹ is O or S;
X² is a bond or C₁₋₃alkyl;
P is C₃₋₇cycloalkyl or C₄₋₇cycloalkenyl;
R¹ is hydrogen, C₁₋₆alkyl, cyano, halogen and C₁₋₆alkylhalo, and one or more R¹ may be connected to each other or to one of the atoms that constitutes P to form a bridge or spirocyclo;
R² is hydrogen, C₁₋₃alkyl, fluoromethyl, difluoromethyl, trifluoromethyl, methoxy, fluoromethoxy, difluoromethoxy or trifluoromethoxy;
R⁴ is hydrogen;
Q is a ring containing 4, 5, 6 or 7 atoms independently selected from C, S, O and N, which may be saturated or partially unsaturated and said ring may further contain groups independently selected from SO, SO₂, CO, cyano and CS;
R³ is hydrogen, hydroxy, halogen, nitro, OC₁₋₃alkylhalo, C₁₋₃alkylhalo, C₁₋₃alkyl, C₁₋₃alkoxyC₀₋₃alkyl, C₁₋₃alkanol, cyano, amino or amide;
n
is 0, 2, 3or4; and
m is 0, 1, 2, 3or4;
or N₁-oxides, salts, solvates or solvated salts thereof.

Salts for use in pharmaceutical compositions will be pharmaceutically acceptable salts, but other salts may be useful in the production of the compounds of formula I.

A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.) 1990.

Some compounds of formula I may have chiral centres and/or geometric isomeric centres (E- and Z- isomers), and it is to be understood that the invention encompasses all such optical, diastereoisomeric and geometric isomers.

The invention relates to compounds of formula I having a trans-relationship between R¹ and X² on ring P, when P is cyclohexane and R¹ and X² is attached to P at position 4 and 1 respectively.

The invention also relates to any and all tautomeric forms of the compounds of formula I.

Pharmaceutical Composition

According to one aspect of the present invention there is provided a pharmaceutical composition comprising as active ingredient a therapeutically effective amount of the compound of formula I, or N₁-oxides, salts, solvates or solvated salts thereof, in association with one or more pharmaceutically acceptable diluent, excipients and/or inert carrier.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers.

Suitable daily doses of the compounds of formula I in the treatment of a mammal, including man are approximately 0.01 to 250 mg/kg bodyweight at peroral administration and about 0.001 to 250 mg/kg bodyweight at parenteral administration.

The typical daily dose of the active ingredients varies within a wide range and will depend on various factors such as the relevant indication, severity of the illness being treated, the route of administration, the age, weight and sex of the patient and the particular compound being used, and may be determined by a physician.

Medical Use

It has been found that the compounds according to the present invention, or N₁-oxides, salts, solvates or solvated salts thereof, exhibit a high degree of potency and selectivity for individual metabotropic glutamate receptor (mGluR) subtypes. Accordingly, the compounds of the present invention are expected to be useful in the treatment of conditions associated with excitatory activation of an mGluR Group I receptor and for inhibiting neuronal damage caused by excitatory activation of an mGluR Group I receptor. The compounds may be used to produce an inhibitory effect of mGluR Group I, in mammals, including man.

The mGluR Group I receptor is highly expressed in the central and peripheral nervous system and in other tissues. Thus, it is expected that the compounds of the invention are well suited for the treatment of mGluR Group I receptor-mediated disorders such as acute and chronic neurological and psychiatric disorders, gastrointestinal disorders, and chronic and acute pain disorders.

The invention relates to compounds of formula I as defined hereinbefore, for use in therapy.

The invention relates to compounds of formula I as defined hereinbefore, for use in treatment of mGluR Group I receptor-mediated disorders.

The invention relates to compounds of formula I as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula I as defined hereinbefore, for use in treatment of pain related to migraine, inflammatory pain, neuropathic pain disorders such as diabetic neuropathies, arthritis and rheumatitiod diseases, low back pain, post-operative pain and pain associated with various conditions including angina, renal or billiary colic, menstruation, migraine and gout.

The invention relates to compounds of formula I as defined hereinbefore, for use in treatment of stroke, head trauma, anoxic and ischemic injuries, hypoglycemia, cardiovascular diseases and epilepsy.

The present invention relates also to the use of a compound of formula I as defined hereinbefore, in the manufacture of a medicament for the treatment of mGluR Group I receptor-mediated disorders and any disorder listed above.

One embodiment of the invention relates to the use of a compound according to formula I in the treatment of gastrointestinal disorders.

Another embodiment of the invention relates to the use of a compound according to formula I, for the manufacture of a medicament for the inhibition of transient lower esophageal sphincter relaxations, for the treatment of GERD, for the prevention of reflux, for the treatment regurgitation, treatment of asthma, treatment of laryngitis, treatment of lung disease and for the management of failure to thrive.

The invention also provides a method of treatment of mGluR Group I receptor-mediated disorders and any disorder listed above, in a patient suffering from, or at risk of, said condition, which comprises administering to the patient an effective amount of a compound of formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

Non-Medical Use

In addition to their use in therapeutic medicine, the compounds of formula I, or N₁-oxides, salts, solvates or solvated salts thereof, are also useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutics agents.

Methods of Preparation

Another aspect of the present invention provides processes for preparing compounds of formula I, or N₁-oxides, salts, solvates or solvated salts thereof.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P.G.M. Wuts, Wiley-Interscience, New York, (1999) (Ref. 1). References and descriptions of other suitable reactions are described in textbooks of organic chemistry, for example, “Advanced Organic Chemistry”, March, 4^{th} ed. McGraw Hill (1992) (Ref. 2) or, “Organic Synthesis”, Smith, McGraw Hill, (1994) (Ref. 3). For representative examples of pyrazine chemistry see for example “Heterocyclic Chemistry”, J. A. Joule, K. Mills, G. F. Smith, 3^{rd} ed. Chapman and Hall (1995), p. 189-224 (Ref. 4) and “Heterocyclic Chemistry”, T. L. Gilchrist, 2^{nd} ed. Longman Scientific and Technical (1992), p. 248-282 (Ref. 5).

The term “room temperature” and “ambient temperature” shall mean, unless otherwise specified, a temperature between 16 and 25° C.

Methods of Preparation of Intermediates

Processes for the preparation of the intermediates, wherein P, Q, X¹, X², R¹, R², R³, R⁴, m and n are, unless otherwise specified, defined as in formula I, comprises of: 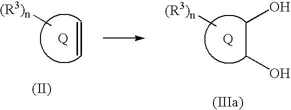
(i-a) reacting a compound of formula II, to obtain a compound of formula IIIa with for example catalytic amounts of osmium tetroxide together with stochiometric amounts of tertiary amine N-oxides, as described by Van Rheenen et al, Tetrahedron Letters (1976), Vol. 17, p. 1973 or as described in Ref. 2,
or,
(i-b) Pinacol cyclization of an open chain dialdehyde to give the compound of formula IIIa as described in Ref. 2, 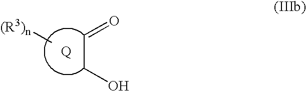
or alternatively,
(i-c) acyloin condensation of an open chain diester, as described in Ref. 2, to give a compound of formula IIIb, 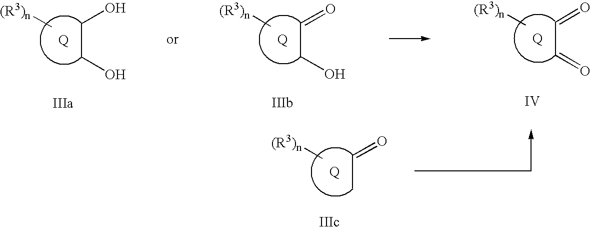
(ii) oxidation of compounds of formula IIIa or IIIb by methods known to the skilled artisan, e.g. Swern oxidation (Ref. 1 or 2), to obtain a compound of formula IV. Alternatively by alpha-oxidation of compounds of formula IIIc according to for example Hunter et al. Tetrahedron Letters (1984), Vol. 25, p 603-606 to yield compounds of formula IV. The R³ group or groups for all compounds may be suitably manipulated by e.g. protection or may be introduced during any of the steps towards the preparation of compounds of formula I, 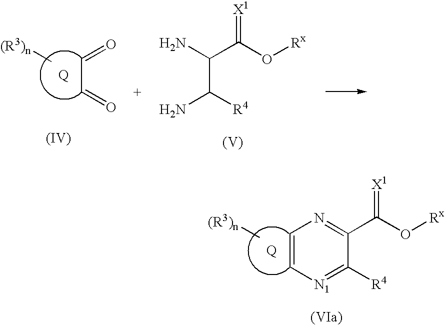
(iii-a) reacting the compound of formula IV, in a suitable solvent such as dichloromethane, acetonitrile, DMF, water or an alcohol such as methanol, or preferably in diethylether or benzene, with a compound of formula V, wherein X¹ is CH₂, O or S and R^{x} is C₁₋₆ alkyl, which may be branched, e.g. tert-butyl, or substituted phenyl or benzyl, in the form of a free base or in the form of a salt such as hydrochloride or hydrobromide, whereas in the latter case the compound of formula V is first neutralized using an appropriate base such as sodium or potassium hydroxide, triethylamine or diisopropyl ethylamine in a suitable solvent such as an alcohol, e.g. methanol, or acetonitrile, dichloromethane, or preferably in diethylether or benzene, in the optional presence of a Lewis acid or a drying agent such as molecular sieves, at a temperature between −20 to 120° C. depending on the solvent employed and the nature of the substituents, to obtain after spontaneous aromatization in the presence of oxygen a compound of formula VIa, as described e.g. in Jones et al., J. Med. Chem., (1998), Vol. 41, p. 3062,
alternatively, if spontaneous aromatization does not take place, further oxidation of the corresponding condensation product may be carried out in the presence of metal salts, according to, for example the method described by Kobayashi et al, Tetrahedron, (1999), Vol. 55, p. 13179,
or,
(iii-b) reacting a cyclic epoxide with a diamine compound of formula V under oxidative conditions to obtain the compound of formula VIa, as described by Antoniotti and Duñach, Tetrahedron Letters, (2002), Vol. 43, p. 3971, whereby X¹ may be carried on the compound of formula V before the condensation, or introduced on the compound of formula VIa by treatment with a suitable reagent such as phosphorous pentasulfide when X¹═O (Ref. 2 or 3) or by oxidation under e.g. oxidative ozonolysis conditions when X¹═CH₂ (Ref. 2 or 3), 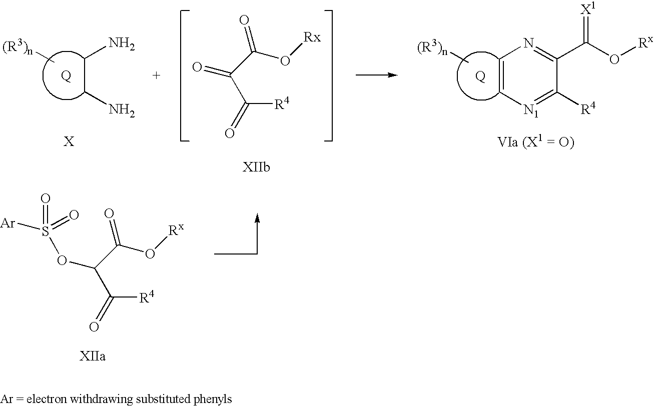
or,
(iii-c) condensation of diamines of formula X in suitable aprotic solvents such as benzene, dichloromethane or diethylether with tricarbonyls of formula XIIb, generated in situ from a compound of formula XIIa by treatment with a base, to yield compounds of formula VIa, wherein X¹ is oxygen, according to, for example Hoffman et el. J. Org. Chem., (1990), Vol. 55, p. 2820-2822, 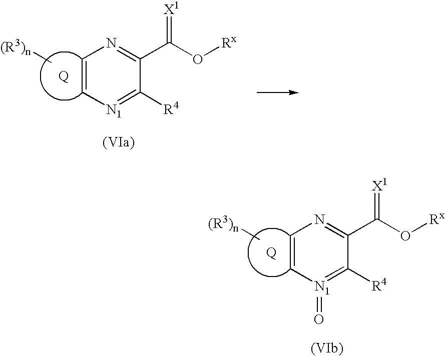
(iv) reacting the compound of formula VIa with an appropriate oxidant such as mCPBA to give a N₁-oxidated compound of formula VIb. This may also be done at a latter stage of the synthesis depending on the compatibility of functional groups present, 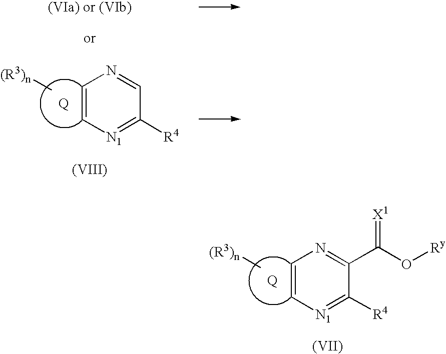
(v-a) reacting the compound of formula VIa or VIb, wherein X¹ and R^{x} are defined as above, in the presence of a strong base, e.g. potassium or lithium hydroxide, or acid, e.g. hydrochloric acid, to yield a compounds of formula VII, wherein R^{y} is H or as the salt of the compound of formula VII, wherein R^{y} is either an organic or an inorganic cation such as the sodium or potassium,
or,
(v-b) reacting a compound of formula VIII, when R⁴ is hydrogen, with a suitable alkyl lithium reagent such as methyl lithium or butyl lithium followed by reaction with carbon dioxide, to obtain the compound of formula VII, or an alkyl chloroformiate or alkyl cyanoformiate to obtain compounds of formula VIa, as described for example by Yves, Fort et al., J. Org. Chem., (2002), Vol. 67, p. 234, for the analogous reactions of pyridines,
or
(v-c) carrying out a Minisci reaction on the compound of formula VIII with oxalic acid mono-esters to give compounds of formula VIa according to, for example Coppa et al. Tetrahedron Letters, (1992), Vol. 33, p.3057-3060, 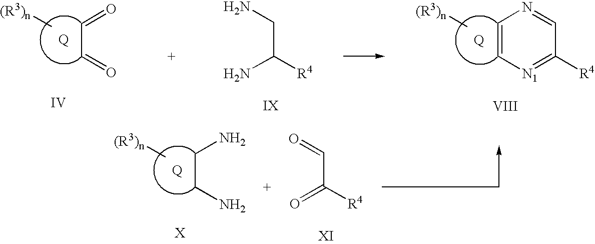
(vi) compounds of formula VIII may be obtained by reacting the compound of formula IV with a compound of formula IX, or X with XI, in a suitable solvent such as
dichloromethane, acetonitrile, DMF, water, diethyl ether, benzene, or an alcohol such as methanol as described by e.g. Justus K. Landquist, J. Chem. Soc., (1953), p. 2816, 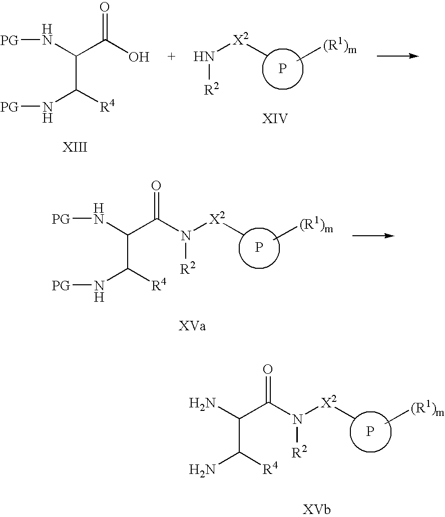
(vii) compounds of formula XVb may be obtained by coupling suitably N-protected aminoacids of formula XIII with amines of formula XIV, using standard procedures for amide formation, such as employment of stochiometric amounts of N,N′-dicyclohexylcarbodiimide together with catalytic amounts of 1-hydroxybezotriazole in DMF at 0-50° C., followed by removal of the protecting groups (PG in drawing) as described in Ref 1. Example of a preferred protecting group is BOC,
(viii) amines of formula XIV, carrying hydrogen as R², can be prepared in two steps from the corresponding aldehydes or ketones via condensation with hydroxylamine, followed by reduction of the resulting oxime using for example sodium in refluxing ethanol. Amines of formula XIV can alternatively be prepared through a reductive amination of the corresponding aldehyde or ketone.
   Preparation of Final Compounds of Formula I

Another object of the invention are processes for the preparation of the compounds of formula I, wherein P, Q, X¹, X², R¹, R², R³, R⁴, m and n are, unless otherwise specified, defined as in formula I, comprising of: 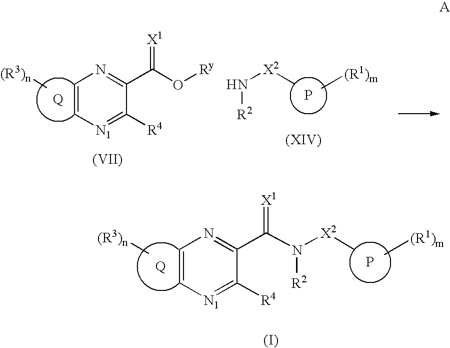
reacting a compound of formula VII, wherein R^{y} is H, with an activating agent, such as oxolyl chloride or thionyl chloride, followed by the treatment of the resulting acid halide, or otherwise to nucleophiles activated acid derivative, with an amine of formula XIV, in the presence of a non-nucleophilic base such as triethylamine or diisopropylethylamine in an appropriate solvent such as methylene chloride or diethylether, to obtain the compound of formula I,
alternatively, 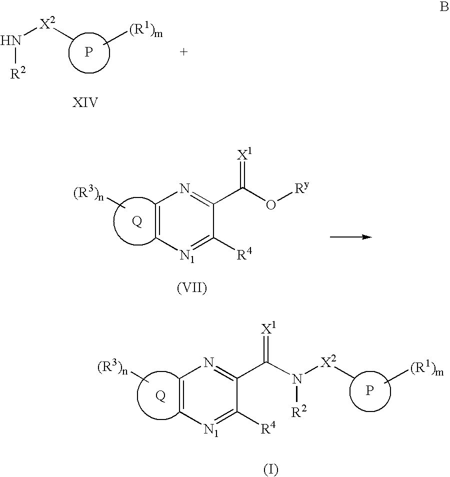
   reacting an amine of formula XIV with the compound of formula VII, wherein R^{y} is H, in the presence of a suitable water abstracting coupling reagent such as HBTU or EDC, with or without a nucleophilic catalyst such as hydroxy benzotriazole, using a non-nucleophilic base such as triethylamine or diisopropyl ethyl amine in an appropriate solvent such as acetonitril or DMF, or other standard methods for amide couplings, to obtain the compound of formula I, or 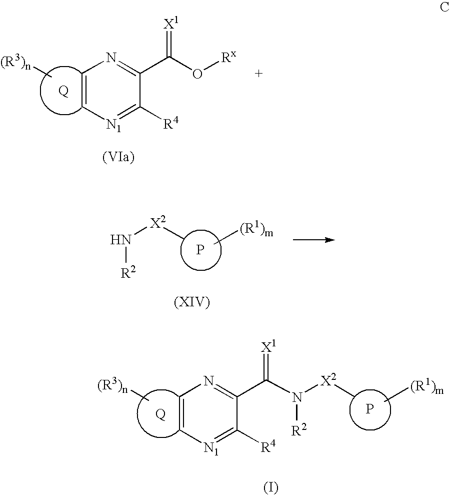
reacting a compound of formula VIa or the N₁-oxide thereof (VIb), wherein R^{x} is
C₁₋₆ alkyl, which may be branched as in e.g. tert-butyl, or a substituted phenyl or benzyl, in the form of a free base or in the form of a salt such as hydrochloride or hydrobromide, with the appropriate amine such as the compound of formula XIV, to obtain the compound of formula I. The reaction may be performed neat or using a suitable solvent such as N,N-dimethylformamide, methylene chloride or ethyl acetate at a temperature ranging from ambient temperature to +150° C. The reaction may be aided by using a base such as potassium carbonate, triethylamine or 1,8-diazabicyclo[5.4.0]undec-7-ene or an acid such as trimethylaluminum or p-toluenesulfonic acid,
or, 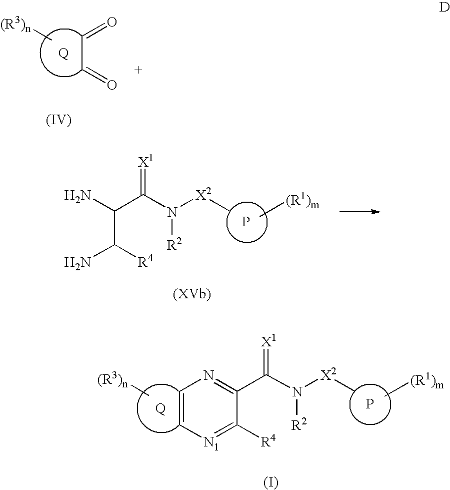
direct condensation of intermediates of formula IV and XVb analogously to the above described formation of intermediates of formula VIa from IV and V, to obtain the compound of formula I.

The invention further relates to compounds of formula VII, XVb, and XIV, which may be used as intermediates in the preparation of the compound of formula I.

One aspect of the invention relates to the compound of formula VII, 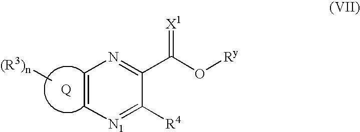
wherein Q, R³, R⁴, X¹ and n are defined as hereinbefore and R^{y} is H.

Another aspect of the invention relates to the compound of formula XIV or salts thereof, 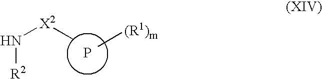
wherein P, R¹, R², X² and m are defined as hereinbefore.

A further aspect of the invention relates to the compound of formula XVb or salts thereof, 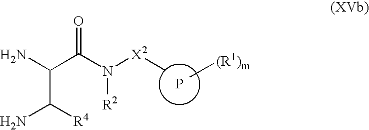
wherein P, R¹, R², R⁴, X² and m are defined as hereinbefore.

The invention further relates to the following compounds, which may be used as intermediates in the preparation of the compound of formula I;
3-methyl-5,6,7,8-tetrahydro-quinoxaline-2-carboxylic acid ethyl ester,
3-methyl-5,6,7,8-tetrahydro-quinoxaline-2-carboxylic acid,
2,3-diamino-N-(4-methyl-cyclohexyl)-propionamide,
4-(tert-butyl-diphenyl-silanyloxy)-cyclohexane-1,2-dione,
6,7-dimethyl-6,7-dihydro-5H-cyclopentapyrazine-2-carboxylic acid methyl ester,
5,6,7,8-tetrahydro-quinoxaline-2-carboxylic acid methyl ester and
5,6,7,8-tetrahydro-quinoxaline-2-carboxylic acid.

### EXAMPLES

The invention will now be illustrated by the following non-limiting examples.

General Methods

All starting materials are commercially available or earlier described in the literature. The ¹H and ¹³C NMR spectra were recorded either on a Brucker 400 or a Varian 400 at 400 MHz and 100 MHz, respectively. The mass spectra were recorded utilising electrospray (LC-MS; LC:Waters 2790, column XTerra MS C₈ 2.5 μm 2.1×30 mm, buffer gradient H₂O+0.1% TFA:CH₃CN+0.04% TFA, MS: micromass ZMD) ionisation techniques.

### Example 1

### N-(trans-4-methylcyclohexyl)-5,6,7,8-tetrahydroquinoxaline-2-carboxamide

5,6,7,8-tetrahydro-quinoxaline-2-carboxylic acid (100 mg) was dissolved in dichloromethane (25 ml). trans-Methyl-cyclohexylamine HCl (83.6 mg) along with catalytic amounts of DMAP (spatula tip) were added and then cooled on an ice/water bath. EDC (113.1 mg) and triethylamine (80 μl) were added. After warming to room temperature the mixture was stirred under nitrogen for 18 h. After addition of a few drops of aquous HCl the mixture was washed twice with water, once with dilute sodium hydroxide solution, followed by brine. Separation of the organic layer and drying over sodium sulfate yielded a white solid after concentration which was purified over 4 g silica with heptane/ethyl acetate (1/1), yielding 41.8 mg (27% ) of N-(trans-4-methylcyclohexyl)-5,6,7,8-tetrahydroquinoxaline-2-carboxamide. ¹H NMR (CDCl₃) δ 9.08 (s, 1 H), 7.58 (brd, 1 H), 3.94-3.81 (m, 1 H). 2.99 (br, 2 H), 2.93 (br, 2 H), 2.09-1.86 (m, 6 H), 1.79-1.67 (br, 2 H), 1.40-1.05 (m, 5 H), 0.89 (d, 3 H). ¹³C NMR (CDCl₃) δ 162.6, 156.1, 151.0, 141.5, 140.9, 48.3, 33.8, 33.0, 32.11, 31.9, 31.8, 22.4, 22.3, 22.2. MS(ES) m/z 274 (M+1).

### 5,6,7,8-Tetrahydro-quinoxaline-2-carboxylic acid methyl ester

Potassium hydroxide (150 mg) was dissovled in methanol (1 ml) and added to a solution of 2,3-diamino-propionic acid methyl ester dihydrochloride in methanol (1 ml). After ultrasonification the mixture was filtered into a solution of 1,2-cyclohexadione (112 mg) in methanol (2 ml). To this solution a few beads of 4 Å MS were added and then heated to reflux for 3 h. After cooling to ambient temperature the molecular sieves were removed via filtration. Evaporation to dryness gave a solid, which was taken up into water and diethyl ether and extracted. The aqueous layer was extracted additionally 4 times with diethyl ether. The organic layers were collected, dried over magnesium sulfate and evaporated to dryness. Purification by chromatography on silica using ethyl acetate/heptane (1/2) yielded 46.4 mg (25% ) of 5,6,7,8-tetrahydro-quinoxaline-2-carboxylic acid methyl ester. ¹H NMR (CDCl₃) δ 8.96 (s, 1 H), 3.94 (s, 3 H), 2.99 (br m, 4 H). 1.91 (br m, 4 H). ¹³C NMR (CDCl₃) δ 194., 157.2, 153.1, 142.9, 139., 52.8, 32.2, 31.9, 22.2, 22.0. MS(ES) m/z 193 (M+1).

### 5,6,7,8-Tetrahydro-quinoxaline-2-carboxylic acid

Method 1

Sodium hydroxide (76.2 mg) was dissolved in methanol (3 ml) and added to a solution of 5,6,7,8-tetrahydro-quinoxaline-2-carboxylic acid methyl ester (169 mg) in methanol (8 ml) followed by stirring at room temperature for 23 h. 1 N HCl (190 ml) and water (50 ml) was added followed by evaporation of methanol. The aquous mixture was extracted three times with diethyl ether. After combining the organic layers, drying over sodium sulfate and evaporation to dryness a white solid was obtained which was further dried under vacuum, yielding 100 mg (46% ) of 5,6,7,8-tetrahydro-quinoxaline-2-carboxylic acid. ¹H NMR (CDCl₃) δ 9.16 (s, 1 H), 3.08 (br, 2 H), 3.01 (br, 2 H). 1.98 (br m, 4 H). MS(ES) m/z 179 (M+1).

Method 2

n-BuLi (80 ml) was cooled on an ice/water bath under an argon atmosphere and a solution of N,N-dimethylaminoethanol (10.0 ml) in anhydrous hexane (80 ml) was added dropwise during 20 min. The mixture was then cooled on an ethanol/dry ice bath after which a solution of 5,6,7,8-tetrahydroquinoxaline (6.3 g) in hexane (40 ml) was added. After 1 h at −75-−78° C. the reaction was poured over on dry ice in diethyl ether. The reaction was kept at −75-−78° C. for 30 min after which 1 M HCl_{(aq)} was added until pH 5 was reached and the slurry was warmed to room temperature. The aqueous layer was separated and extracted with diethyl ether twice, the ether phases were combined and extracted with aqueous sodium hydrogen carbonate, which was then acidified and extracted three times with diethyl ether. The organic phases were combined, dried over sodium sulfate, filtered and evaporated to dryness. Silica gel chromatography using a gradient from heptane/ethylacetate 4/1 to neat ethyl acetate to neat methanol yielded 1.3 g, 15% of 5,6,7,8-tetrahydro-quinoxaline-2-carboxylic acid.

### Example 2

### N-(4,4-dimethylcyclohexyl)-5,6,7,8-tetrahydroquinoxaline-2-carboxamide

Potassium hydroxide (47.2 mg) and 5,6,7,8-tetrahydro-quinoxaline-2-carboxylic acid methyl ester (30 mg) were dissolved in methanol (5 ml) followed by stirring at room temperature for 18 h. The reaction mixture was then evaporated to dryness, dissolved in DMF (2 ml) and treated with trifluoroacetic acid (0.76 ml). To this mixture was added ethyl diisopropylamine (0.35 ml) followed by amine 4,4-dimethyl-cyclohexylamine HCl (33.2 mg), HBTU (60.2 mg) and additional DMF (1 ml). After stirring at room temperature under nitrogen for 19 h the reaction mixture was evaporated to dryness. Dilution with dichloromethane (20 ml), washing with dilute HCl, followed by 1 M NaOH and brine and subsequent drying of the organic layer over sodium sulfate, gave after evaporation to dryness, a crude material, which was purified by chromatography on silica using heptane/ethyl acetate (3/1). This yielded 31 mg (72% ) of the title compound as yellow oil. ¹ H NMR (CDCl₃) δ 9.09 (s, 1 H,), 7.66 (brd, 1 H), 3.95-3.85 (m, 1 H,). 2.99 (br, 2 H,), 2.94 (br, 2 H,), 1.98-1.79 (m, 8 H,), 1.54-1.20 (m, 5 H,),0.93 (d, 6 H). ¹³C NMR (CDCl₃) δ 162.7, 156.1, 151.0, 141.5, 140.9, 48.3, 37.6, 32.1, 31.8, 29.5, 28.7, 22.4, 22.3. MS(ES) m/z 288 (M+1).

### Example 3

### N-(4,4-dimethylcyclohexyl)-3-methyl-5,6,7,8-tetrahydroquinoxaline-2-carboxamide

A solution of 3-methyl-5,6,7,8-tetrahydro-quinoxaline-2-carboxylic acid (10.5 mg), HBTU (25 mg) and diisopropyl ethyl amine (28.5 μl) in DMF (1 ml)was stirred for 5 min. 4,4-Dimethyl-cyclohexylamine hydrochloride (10 mg) was then added and the resulting solution was stirred 3 h under a nitrogen atmosphere. The reaction mixture was diluted with 2 ml water and extracted twice with EtOAc. The combined organic phases were washed with 1M HCl, NaHCO₃₍ₛₐₜ₎, water and brine, dried and concentrated. Flashchromatography (SiO₂, heptane/EtOAc 4:1) afforded 4.1 mg of the title compound. ¹H NMR (CDCl₃) δ 7.85 (bd, 1 H) 3.79-3.89 (m, 1 H) 2.91-2.96 (m, 4 H) 2.90 (s, 3 H)1.92 (m, 4 H) 1.84 (m, 2 H) 1.22-1.50 (m, 8 H) 0.95 (s, 3 H) 0.93 (s, 3 H). MS(ES) m/z 302 (M+1)

### 3-Methyl-5,6,7,8-tetrahydro-quinoxaline-2-carboxylic acid ethyl ester

Ethylacetoacetate (253 μl) was added to a mixture of [hydroxy(2,4-dinitrobenzenesulfonyloxy)iodo]benzene (1030 mg) (ref. Koser, G. F.; Wettach, R. H.; J. Org. Chem. 42, 8, (1977), p 1476-1478) in acetonitrile (28 ml). The reaction mixture was heated at reflux for 1 h. The reaction mixture was cooled and concentrated. The crude product was washed with hexane and then dissolved in benzene (25 ml). Triethyl amine (1 ml) was added and the resulting red solution was strirred for 45 min. pTsOH (10 mg) was added followed by 1,2-aminocyclohexane (490 μl) (cis/trans mixture). The reaction mixture was stirred overnight at room temperature and then concentrated. The crude product was left to stand in air for 24 h. The product was purified by flashchromatography (SiO₂, heptane/EtOAc 6:1) followed by prep-HPLC to afford 13.5 mg of the desired product. ¹H NMR (CDCl₃) δ 4.45 (q, 2 H) 2.96 (m, 4 H) 2.73 (s, 3 H) 1.92 (m, 4 H) 1.41 (t, 3 H)

### 3-Methyl-5,6,7,8-tetrahydro-quinoxaline-2-carboxylic acid

LiOH hydrate (3.9 mg) was added to a solution of 3-methyl-5,6,7,8-tetrahydro-quinoxaline-2-carboxylic acid ethyl ester (13.5 mg) in THF/water 1:1 (2 ml) and the resulting solution was stirred overnight at room temperature. The reaction mixture was acidified with 1M HCl and then extracted with ether and EtOAc. The organic phase was dried and concentrated to give 10.5 mg of the acid which was used directly in the next step.

### Example 4

### 8-methyl-N-(trans-4-methylcyclohexyl)-5,6,7,8-tetrahydroquinoxaline-2-carboxamide

2,3-Diamino-N-(4-methyl-cyclohexyl)-propionamide (100 mg) and 3-methyl-cyclohexane-1,2-dione (63 mg) with 10 mg PPTS (10 mg) were dissolved in benzene (50 ml). The solution was refluxed overnight and then concentrated under reduced pressure.

Flashchromatography (SiO₂, heptane/EtOAc 4:1) afforded 32 mg. ¹H NMR (CDCl₃) δ 9.08 (s, 1 H) 7.57 (bd, 1 H) 3.88 (m, 1 H) 3.00 (m, 3 H) 2.04 (m, 4 H) 1.86 (m, 1 H) 1.75 (m, 2 H) 1.63 (m, 1 H) 1.37 (d, 3 H) 1.28 (m, 2 H) 1.10 (m, 2 H) 0.92 (d, 3 H). MS(ES) m/z 2.88 (M+1)

### 2 3-diamino-N-(4-methyl-cyclohexyl)-propionamide

BOC₂O (17.1 g) was added to a solution of D,L-2,3-diaminopropionic acid (5.0 g) and triethylamine (24.8 ml) in dioxane/water 1:1 (76 ml). The solution was stirred overnight. EtOAc (100 ml) was added and the water phase was acidified with 1M HCl. The phases were separated and the organic phase was washed with brine, dried and concentrated to a give 8.5 g of 2,3-bis-tert-butoxycarbonylamino-propionic acid as a white solid. 6.0 g of the product was dissolved in DMF (180 ml) with HBTU (8.95 g) and diisopropylethylamine (10.2 ml). The resulting solution was stirred for 10 min before 4,4-dimethyl-cyclohexylamine hydrochloride (3.25 g) was added. The solution was stirred for 3.5 h at room temperature and then diluted with 250 ml CH₂Cl₂. The mixture was washed with 1M HCl, water, NaHCO₃₍ₛₐₜ₎ and brine, dried and concentrated to give 7.63 g of a crude product which was used without further purification.

7.0 g of the coupling product was added to 30 ml of an ice-cold 2.5M HCl/MeOH solution. The mixture was then stirred at room temperature for 6 h before being neutralized with 1M NaOH. The aqueous solution was then extracted with CH₂Cl₂. The organic phase was dried and concentrated to give 2.66 g. ¹H NMR (DMSO) δ 7.62 (bd, 1 H) 2.98 (m, 1 H) 2.64 (m, 1 H) 1.72 (m,2 H)1.63 (m,2 H) 1.28 (m, 1 H) 1.15 (m, 2 H) 0.94 (m, 2 H) 0.84 (d, 3 H)

### Example 5

### 7-hydroxy-5,7-dimethyl-N-(trans-4-methylcyclohexyl)-6,7-dihydro-5H-cyclopenta[b]pyrazine-2-carboxamide

2,3-Diamino-N-(4-methyl-cyclohexyl)-propionamide (100 mg) and 3,5-dimethyl-cyclopentane-1,2-dione (63 mg) with PPTS (10 mg) were dissolved in benzene (50 ml). The solution was refluxed overnight and then concentrated under reduced pressure. Flashchromatography (SiO₂, heptane/EtOAc 1:1) afforded 6.5 mg. ¹H NMR (CDCl₃) δ 9.17 (s, 1 H) 7.63 (bd, 1 H) 3.90 (m, 1 H) 3.43 (m, 1 H) 2.65 (m, 1 H) 2.00-2.11 (m, 2 H) 1.80-1.85 (m, 1 H) 1.73-1.79 (m, 2 H) 1.70 (s, 3 H) 1.39 (d, 3 H) 1.24-1.36 (m, 3 H) 1.06-1.17 (m, 2 H) 0.92 (d, 3 H). MS(ES) m/z 304 (M+1)

### Example 6

### N-(trans-4-methylcyclohexyl)-6,7,8,9-tetrahydro-5H-cyclohepta[b]pyrazine-2-carboxamide

2,3-Diamino-N-(4-methyl-cyclohexyl)-propionamide (106 mg), cycloheptane-1,2-dione (67 mg) and PPTS (13 mg) in benzene (10 ml) was refluxed overnight. After cooling the solvent was removed under reduced pressure and the residue was left to stand in air for 72 h. Flashchromatography (SiO₂, heptane/EtOAc 4:1) afforded 86 mg. ¹H NMR (CDCl₃) δ 8.99 (s, 1 H) 7.59 (bd, 1 H) 3.81-3.91 (m, 1 H) 3.00-3.08 (m, 4 H) 1.98-2.05 (m, 2 H) 1.90 (m, 2 H) 1.66-1.76 (m, 6 H) 1.29-1.40 (m, 1 H) 1.21-1.29 (m, 2 H) 1.02-1.14 (m, 2 H) 0.88 (d, 3 H). MS(ES) m/z 288 (M+1)

### Example 7

### 7-methyl-N-(trans-4-methylcyclohexyl)-5,6,7,8-tetrahydroquinoxaline-2-carboxamide and

### 6-methyl-N-(trans-4-methylcyclohexyl)-5,6,7,8-tetrahydroquinoxaline-2-carboxamide

2,3-Diamino-N-(4-methyl-cyclohexyl)-propionamide (150 mg), 4-methyl-cyclohexane-1,2-dione (95 mg) and PPTS (13 mg) in benzene (10 ml) was refluxed overnight. After cooling the solvent was removed under reduced pressure and the residue was left to stand in air for 72 h. Flashchromatography (SiO₂, heptane/EtOAc 4:1) afforded 19 mg of isomer 1 and 10 mg of isomer 2.

Isomer 1: ¹H NMR (CDCl₃) δ 9.09 (s, 1 H) 7.58 (d, 1 H) 3.83-3.93 (m, 1 H) 3.05-3.11 (m, 1 H) 2.92-3.02 (m, 2 H) 2.60 (dd, 1 H) 1.98-2.07 (m, 4 H) 1.70-1.79 (m, 2 H) 1.55 (m, 1 H) 1.32-1.42 (m, 1 H) 1.27 (m, 2 H) 1.13 (d, 3 H) 1.05-1.11 (m, 3 H) 0.91 (d, 3 H). MS(ES) m/z 288 (M+1)

Isomer 2: ¹H NMR (CDCl₃) δ 9.09 (s, 1 H) 7.58 (d, 1 H) 3.83-3.94 (m, 1 H) 2.93-3.12 (m, 3 H) 2.56 (dd, 1 H) 2.03 (m, 4 H) 1.70-1.79 (m, 2 H) 1.49-1.59 (m, 1 H) 1.33-1.38 (m, 1 H) 1.24-1.31 (m, 2 H) 1.13 (d, 3 H) 1.05-1.13 (m, 2 H) 0.91 (d, 3 H. MS(ES) m/z 288 (M+1)

### Example 8

### N-(trans-4-methylcyclohexyl)-6,7-dihydro-5H-cyclopenta [b]pyrazine-2-carboxamide

A solution of 2,3-diamino-N-(4-methyl-cyclohexyl)-propionamide (102 mg), cyclopentane-1,2-dione (50 mg) and PPTS (13 mg) in benzene (10 ml) was refluxed overnight. After cooling the solvent was removed under reduced pressure and the residue was left to stand in air for 72 h. Flashchromatography (SiO₂, heptane/EtOAc 4:1) afforded 2.4 mg. ¹H NMR (CDCl₃) δ 9.09 (s, 1 H) 7.62 (s, 1 H) 3.84-3.95 (m, 1 H) 3.07 (m, 3 H) 2.20-2.31 (m, 2 H) 2.00-2.09 (m, 2 H) 1.70-1.81 (m, 2 H) 1.24-1.34 (m, 3 H) 1.05-1.17 (m, 2 H) 0.91 (d, 3 H). MS(ES) m/z 260 (M+1)

### Example 9

### N-(trans-4-methylcyclohexyl)-7,8-dihydro-5H-pyrano [3,4-b]pyrazine-2-carboxamide

A solution of 2,3-diamino-N-(4-methyl-cyclohexyl)-propionamide (154 mg), 4-hydroxy-6H-pyran-3-one (88 mg) and PPTS (20 mg) in benzene (15 ml) was stirred at room temperature overnight. The solvent was removed under reduced pressure and the residue was left to stand in air for 72 h. Flashchromatography (SiO₂, heptane/EtOAc 4:1) afforded 9.6 mg N-(trans-4-methylcyclohexyl)-7,8-dihydro-5H-pyrano[3,4-b]pyrazine-2-carboxamide and 11.8 mg N-(trans-4-methylcyclohexyl)-7,8-dihydro-5H-pyrano[3,4-b]pyrazine-3-carboxamide.

N-(trans-4-methylcyclohexyl)-7,8-dihydro-5H-pyrano[3,4-b]pyrazine-2-carboxamide: ¹H NMR (CDCl₃) δ 9.15 (s, 1 H) 7.56 (bd, 1 H) 4.86 (s, 2 H) 4.10 (m, 2 H) 3.84-3.95 (m, 1 H) 3.07 (t, 2 H) 2.00-2.08 (m, 2 H) 1.71-1.79 (m, 2 H) 1.34-1.39 (m, 1 H) 1.25-1.32 (m, 2 H) 1.05-1.17 (m, 2 H) 0.91 (d, 3 H). MS(ES) m/z 276 (M+1)

### 4-Hydroxy-6H-pyran-3-one

2,2,6,6-Tetramethyl-1-oxo-piperidinium chloride (383 mg) was added slowly to a solution of tetrahydro-4H-pyran-4-one (200 mg) and pTsOH (7.6 mg) in acetonitrile (2 ml) at 0° C. The reaction mixture was stirred for 30 min when everything had dissolved and the dark orange color had disappeared. The solution was then refluxed for 15 min. After cooling, ether was added and the salts were removed by filtration. The volatile were removed under reduced pressure. Flashchromatography (SiO₂, heptane/EtOAc 4:1) afforded 88 mg. ¹H NMR (CDCl₃) δ 6.14 (t, 1 H) 4.43 (dd, 2 H) 4.24 (d, 2 H)

### Example 10

### N-(trans-4-methylcyclohexyl)-7,8-dihydro-5H-pyrano [3,4-b]pyrazine-3-carboxamide

Prepared as Described in Example 9.

N-(trans-4-methylcyclohexyl)-7,8-dihydro-5H-pyrano[3,4-b]pyrazine-3-carboxamide: ¹H NMR (CDCl₃) δ 9.20 (s, 1 H) 7.49 (bd, 1 H) 4.82 (s, 2 H) 4.11 (m, 2 H) 3.85-3.93 (m, 1 H) 3.13 (t, 2 H) 2.04 (m, 2 H) 1.75 (m, 2 H) 1.33-1.41 (m, 1 H) 1.23-1.32 (m, 1.05-1.16 (m, 2 H) 0.91 (d, 3 H). MS(ES) m/z 276 (M+1)

### Example 11

### 7-hydroxy-N-(trans-4-methylcyclohexyl)-5,6,7,8-tetrahydroquinoxaline-2-carboxamide and

### 6-hydroxy-N-(trans-4-methylcyclohexyl)-5,6,7,8-tetrahydroquinoxaline-2-carboxamide

TBAF (350 μl, 1.0M in THF) was added dropwise to a solution of isomer 1 (106 mg) in THF (5 ml). The reaction mixture was stirred overnight at room temperature. The solution was diluted with water and extracted with EtOAc. The organic phase was washed with brine and water, dried and concentrated. Flashchromatography (SiO₂, heptane/EtOAc 1:1) afforded 9 mg. ¹H NMR (CDCl₃) δ 9.12 (s, 1 H) 7.58 (bd, 1 H) 4.36-4.46 (m, 1 H) 3.83-3.94 (m, 1 H) 3.29 (dd, 1 H) 3.18 (m, 1 H) 3.04 (dd, 1 H) 2.95 (m 1 H) 2.01-2.12 (m, 4 H) 1.71-1.80 (m, 2 H) 1.35-1.39 (m, 1 H) 1.24-1.32 (m, 2 H) 1.05-1.17 (m, 2 H) 0.91 (d, 3 H). MS(ES) m/z 290 (M+1)

TBAF (510 μl, 1.0M in THF) was added dropwise to a solution of isomer 2 (151 mg) in THF (5 ml). The reaction mixture was stirred overnight at room temperature. The solution was diluted with water and extracted with EtOAc. The organic phase was washed with brine and water, dried and concentrated. Flashchromatography (SiO₂, heptane/EtOAc 1:1) afforded 48 mg. ¹H NMR (CDCl₃) δ 9.08 (s, 1 H) 7.58 (bd, 1 H) 4.39 (m, 1 H) 3.86 (m, 1 H) 3.16-3.26 (m, 2 H) 3.00 (m, 2 H) 1.99-2.11 (m, 4 H) 1.74 (m, 2 H) 1.34-1.39 (m,2 H) 1.23-1.33 (m, 2 H) 1.03-1.14 (m, 2 H) 0.90 (d, 3 H). MS(ES) m/z 290 (M+1)

### 4-(tert-Butyl-diphenyl-silanyloxy)-cyclohexane-1,2-dione

767 mg (4 mmol) 2,2,6,6-tetramethyl-1-oxo-piperidinium chloride (767 mg) was added slowly to a solution of 4-(tert-butyl-diphenyl-silanyloxy)-cyclohexanone (1.41 g) and pTsOH (15 mg) in acetonitrile/CH₂Cl₂ 4:1 (25 ml) at 0° C. The reaction mixture was stirred until everything had dissolved. The solution was then heated at 90° C. for 20, cooled and then concentrated. Flashchromatography (SiO₂, heptane/EtOAc 6:1) afforded 970 mg of the product as a mixture of tautomers which was used directly in the next step.

### 7-(tert-Butyl-diphenyl-silanyloxy)-5,6,7,8-tetrahydro-quinoxaline-2-carboxylic acid (4-methyl-cyclohexyl)-amide and

### 6-(tert-Butyl-diphenyl-silanyloxy)-5,6,7,8-tetrahydro-quinoxaline-2-carboxylic acid (4-methyl-cyclohexyl)-amide

A solution of 2,3-diamino-N-(4-methyl-cyclohexyl)-propionamide (500 mg), 4-(tert-butyl-diphenyl-silanyloxy)-cyclohexane-1,2-dione (970 mg, mixture of tautomers) and PPTS (63 mg) in benzene (50 ml)was stirred at room temperature overnight. The solvent was removed under reduced pressure and the residue was left to stand in air for 72 h. Flashchromatography (SiO₂, heptane/EtOAc 4:1) afforded 106 mg of isomer 1 and 151 mg of isomer 2.

Isomer 1: ¹H NMR (CDCl₃) δ 9.11 (s, 1 H) 7.67 (dd, 2 H) 7.60 (dd, 2 H) 7.32-7.43 (m, 6 H) 4.35 (m, 1 H) 3.90 (m, 1 H) 3.18-3.28 (m, 1 H) 3.05 (m, 2 H) 2.84 (m, 1 H) 1.98-2.11 (m, 3 H) 1.84-1.94 (m, 1 H) 1.75 (m, 2 H) 1.29-1.38 (m, 1 H) 1.24-1.28 (m, 3 H) 1.05-1.16 (m, 2 H) 1.01 (s, 9 H) 0.91 (d, 3 H). MS(ES) m/z 528 (M+1)

Isomer 2: ¹H NMR (CDCl₃) δ 9.11 (s, 1 H) 7.67 (dd, 2 H) 7.58 (dd, 2 H) 7.32-7.44 (m, 6 H) 4.34-4.41 (m, 1 H) 3.89 (m, 1 H) 3.21-3.31 (m, 1 H) 2.99 (m, 2 H) 2.89 (m, 1 H) 1.98 −2.09 (m, 3 H) 1.84-1.95 (m, 1 H) 1.70-1.80 (m, 2 H) 1.22-1.33 (m, 3 H) 1.05-1.17 (m, 2 H) 1.01 (s, 9 H) 0.91 (d, 3 H). MS(ES) m/z 528 (M+1)

### Example 12

### N-(4,4-dimethylcyclohexyl)-5,6,7,8-tetrahydroquinoxaline-2-carboxamide 4-oxide

5,6,7,8-Tetrahydro-quinoxaline-2-carboxylic acid (4,4-dimethyl-cyclohexyl)-amide (13.5 mg) and mCPBA (88.0 mg) were dissolved in dichloromethane (2 mL) and stirred at ambient temperatures over night. The reaction mixture was purified on silica using heptane/ethyl acetate 2:1 as eluent. The isolated product was taken up in dichloromethane and washed with aq. Na₂S₂O₄, then with aq. NaHCO₃ and finally with water. After drying over NaSO₄, evaporation and drying in vacuo, the title compound was obtained (4.1 mg, 29% ). ¹H NMR (CDCl₃) δ 8.73 (s, 1H), 7.64 (br d, 1H), 3.96-3.80 (m, 1 H), 2.99-2.84 (br m, 4H), 2.01-1.77 (m, 2H), 1.56-1.26 (m, 5H), 0.94 (d, 6H). ¹³C NMR (CDCl₃) δ 160.9, 155.4, 145.4, 145.3, 131.0, 48.7, 37.6, 31.8, 29.7, 29.6, 28.7, 24.0, 21.7, 21.3. MS(ES) m/z 304 (M+1).

### Example 13

### 6,7-dimethyl-N-(trans-4-methylcyclohexyl)-6,7-dihydro-5H-cyclopenta[b]pyrazine-2-carboxamide

6,7-Dimethyl-6,7-dihydro-5H-cyclopentapyrazine-2-carboxylic acid methyl ester (6 mg) was dissolved in methanol (1.5 ml). IM sodium hydroxide (50 ml) was added and the reaction was stirred at room temperature for 4 h. The solution was acidified with 1 M hydrochloric acid and the methanol was evaporated. The water was washed with diethyl ether and the organic layer was dried, filtered and evaporated.

To the residue, dissolved in DMF (1 ml), was added diisopropyletylamine (25 mL), HBTU (15.5 mg) and trans-4-methylcyclohexylamine (5.2 mg). The reaction was stirred at room temperature for 48 h, evaporated and purified through silica gel chromatography (heptane/ethyl acetate 3:1) to give 4 mg of the product. ¹H.NMR (CDCl₃) δ 9.1 (s, 1 H) 7.6 (d, 1 H) 3.9 (m, 1 H) 3.2 (dd, 1 H) 2.7(dd, 1 H) 1.4 (d, 3H) 1.3(d, 3 H) 0.95 (d, 3 H). MS(ES) m/z 288 (M+1)

### 6,7-Dimethyl-6,7-dihydro-5H-cyclopentapyrazine-2-carboxylic acid methyl ester

A mixture of 2,3-diamino-propionic acid methyl ester di-hydrochloride (90 mg) in methanol (0.5 ml) was treated with a methanolic potassium hydroxide solution (94 mg in 0.5 ml) and stirred vigorously at room temperature for 5 min. The solution was filtered and treated with 3,4-dimethyl-cyclopentane-1,2-dione (64 mg) dissolved in methanol (1 ml). Molecular sieves (4 Å) were added and the reaction refluxed for 40 h.

The mixture was filtered, acidified with IM hydrochloride acid and partitioned between water and diethyl ether. The organic layer was dried, filtered, evaporated and purified by silica gel chromatography (heptane/ethyl acetate 3:1) to give 6 mg of the product. ¹H.NMR (CDCl₃) δ 9.0(s, 1 H) 4.0 (s, 3 H) 3.2(dd, 1 H) 2.7 (dd, 1 H) 1.4 (d, 3 H) 1.3 (d, 3 H). MS(ES) m/z 207 (M+1)

Abbreviations

BOC tert-butoxycarbonyl
nBu normal butyl
mCPBA meta-chloroperoxybenzoic acid
DMAP 4(N,N-dimethylamino)pyridine
DMF dimethylformamide
EDC 1-Ethyl-3-(3-dimethylaminopropyl)carbodiimide
HBTU O-benzotriazol-1-yl-N,N,N′,N′-tetramethyluronium hexafluorophosphate
PPTS pyridinium p-toluenesulfonate
TBAF tetrabutylammonium fluoride
THF tetrahydrofuran
pTsOH p-toluenesulfonic acid
   Pharmacology

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15:6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

For FLIPR analysis, cells expressing human mGluR5d or recombinant mGluR1 as described in WO97/05252 were seeded on collagen coated clear bottom 96-well plates with black sides and analysis of [Ca²⁺]ᵢ mobilization was done 24 h after seeding. FLIPR experiments were done using a laser setting of 0.800 W and a 0.4 second CCD camera shutter speed. Each FLIPR experiment was initiated with 160 μl of buffer present in each well of the cell plate. After each addition of the compound, the fluorescence signal was sampled 50 times at 1 second intervals followed by 3 samples at 5 second intervals. Responses were measured as the peak height of the response within the sample period. EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d or recombinant mGluR1 as described in WO97/05252 based on Inositol Phosphate (IP₃) turnover. IP₃ accumulation is measured as an index of receptor mediated phospholipase C turnover. GHEK cells stably expressing the human mGluR5d or recombinant mGluR1 receptors were incubated with [3H] myo-inositol overnight, washed three times in HEPES buffered saline and pre-incubated for 10 min with 10 mM LiCl. Compounds (agonists) were added and incubated for 30 min at 37° C. Antagonist activity was determined by pre-incubating test compounds for 15 min, then incubating in the presence of glutamate (80 μM) or DHPG (30 μM) for 30 min. Reactions were terminated by the addition of perchloric acid (5% ). Samples were collected and neutralized, and inositol phosphates were separated using Gravity-Fed Ion-Exchange Columns.

A detailed protocol for testing the compounds of the invention is provided in the assay below.

Assay of Group I Receptor Antagonist Activity

For FLIPR analysis, cells expressing human mGluR5d or recombinant mGluR1 as described in WO97/05252 were seeded on collagen coated clear bottom 96-well plates with black sides and analysis of [Ca²⁺]ᵢ mobilization was performed 24 h following seeding. Cell cultures in the 96-well plates were loaded with a 4 μM solution of acetoxymethyl ester form of the fluorescent calcium indicator fluo-3 (Molecular Probes, Eugene, Oreg.) in 0.01% pluronic. All assays were performed in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). FLIPR experiments were done using a laser setting of 0.800 W and a 0.4 second CCD camera shutter speed with excitation and emission wavelengths of 488 nm and 562 nm, respectively. Each FLIPR experiment was initiated with 160 μl of buffer present in each well of the cell plate. A 40 μl addition from the antagonist plate was followed by a 50 μL addition from the agonist plate. After each addition the fluorescence signal was sampled 50 times at 1 second intervals followed by 3 samples at 5 second intervals. Responses were measured as the peak height of the response within the sample period.

EC₅₀/IC₅₀ determinations were made from data obtained from 8 points concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

Measurement of Inositol Phosphate Turnover in Intact Whole Cells

GHEK stably expressing the human mGluR5d or recombinant mGluR1 receptor were seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells were washed once in HEPES buffered saline and pre-incubated for 10 min in HEPES buffered saline containing 10 mM LiCl. Compounds (agonists) were added and incubated at 37° C. for 30 min. Antagonist activity was determined by pre-incubating test compounds for 15 min, then incubating in the presence of glutamate (80 μM) or DHPG (30 μM) for 30 min. The reaction was terminated by the addition of 0.5 ml perchloric acid (5% ) on ice, with incubation at 4° C. for at least 30 min. Samples were collected in 15 ml Falcon tubes and inositol phosphates were separated using Dowex columns, as described below.

Assay For Inositol Phosphates Using Gravity-Fed Ion-Exchange Columns

Preparation of Ion-Exchange Columns

Ion-exchange resin (Dowex AG1-X8 formate form, 200-400 mesh, BIORAD) was washed three times with distilled water and stored at 4° C. 1.6 ml resin was. added to each column, and washed with 3 ml 2.5 mM HEPES, 0.5 mM EDTA, pH 7.4.

a) Sample Treatment

Samples were collected in 15 ml Falcon tubes and neutralized with 0.375 M HEPES, 0.75 M KOH. 4 ml of HEPES/EDTA (2.5/0.5 mM, pH 7.4) were added to precipitate the potassium perchlorate. Supernatant was added to the prepared Dowex columns.

b) Inositol Phosphate Separation

Elute glycero phosphatidyl inositols with 8 ml 30 mM ammonium formate. Elute total inositol phosphates with 8 ml 700 mM ammonium formate/100 mM formic acid and collect eluate in scintillation vials. Count eluate mixed with 8 ml scintillant.

One aspect of the invention relates to a method for inhibiting activation of Group I mGluR receptors, comprising treating a cell containing said receptor with an effective amount of the compound of formula I.

Screening for Compounds Active Against Tlesr

Adult Labrador retrievers of both genders, trained to stand in a Pavlov sling, are used. Mucosa-to-skin esophagostomies are formed and the dogs are allowed to recover completely before any experiments are done.

Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 ml/kg) in a foreleg vein. Ten min after i.v. administration, a nutrient meal (10% peptone, 5% D-glucose, 5% Intralipid, pH 3.0) is infused into the stomach through the central lumen of the assembly at 100 ml/min to a final volume of 30 ml/kg. The infusion of the nutrient meal is followed by air infusion at a rate of 500 ml/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal ≦2s before its onset in which case the relaxation is classified as swallow-induced. The pressure difference between the LES and the stomach should be less than 2 mmHg, and the duration of the complete relaxation longer than 1 s.

Abbreviations

BSA Bovine Serum Albumin
CCD Charge Coupled Device
CRC Concentration Response Curve
DHPG 3,5-dihydroxyphenylglycine;
EDTA Ethylene Diamine Tetraacetic Acid
FLIPR Fluorometric Imaging Plate reader
GHEK GLAST-containing Human Embrionic Kidney
GLAST glutamate/aspartate transporter
HEPES 4-(2-hydroxyethyl)-1-piperazineethanesulfonic acid (buffer)
IP₃ inositol triphosphate
   Results

Typical IC₅₀ values as measured in the assays described above are 10 μM or less. In one aspect of the invention the IC₅₀ is below 2 μM. In another aspect of the invention the IC₅₀ is below 0.2 μM. In a further aspect of the invention the IC₅₀ is below 0.05 μM.

| | mGluR1 | mGluR5 |
|---|---|---|
| | IC₅₀ | IC₅₀ |
| Compound | (nM) | (nM) |
| 6-methyl-N-(trans-4-methylcyclohexyl)-5,6,7,8- | 52 | 7289 |
| tetrahydroquinoxaline-2-carboxamide | | |

1. A compound having the formula I 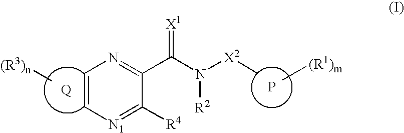
wherein:
X¹ is O or S;
X² is a bond or C₁₋₃alkyl;
P is C₃₋₇cycloalkyl or C₄₋₇cycloalkenyl;
R¹ is hydrogen, C₁₋₆alkyl, cyano, halogen and C₁₋₆alkylhalo, and one or more R¹ may be connected to each other or to one of the atoms that constitutes P to form a bridge or spirocyclo;
R² is hydrogen, C₁₋₃alkyl, fluoromethyl, difluoromethyl, trifluoromethyl, methoxy, fluoromethoxy, difluoromethoxy, trifluoromethoxy, C₀₋₃alkylamino, C₀₋₃alkylhydroxy or C₀₋₃alkyldimethylamino;
R⁴ is hydrogen, C₁₋₃alkyl, fluoromethyl, difluoromethyl, trifluoromethyl, methoxy, fluoromethoxy, difluoromethoxy, trifluoromethoxy, C₀₋₃alkylamino, C₀₋₃alkylhydroxy or C₀₋₃alkyldimethylamino;
Q is a ring containing 4, 5, 6 or 7 atoms independently selected from C, S, O and N, which may be saturated or partially unsaturated and said ring may further contain groups independently selected from SO, SO₂, CO, cyano and CS;
R³ is hydrogen, hydroxy, halogen, nitro, cyano, OC₁₋₃alkylhalo, C₁₋₃alkylhalo, C₁₋₃alkyl, C₁₋₃alkoxyC₀₋₃alkyl, C₀₋₃alkylOC₂₋₄alkanol C₁₋₃alkanol, amino, C₁₋₃alkylaminoC₀₋₃alkyl, (C₁₋₃alkyl)₂aminoC₀₋₃alkyl, amide, C₁₋₃alkylamideC₀₋₃alkyl or (C₁₋₃alkyl)₂amideC₀₋₃alkyl;
n is 0, 1, 2, 3 or 4; and
m is 0, 1, 2, 3 or 4;
or N₁-oxides, salts, solvates or solvated salts thereof.

2. A compound having the formula I 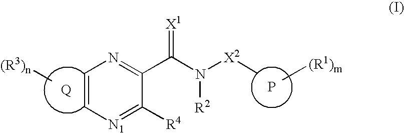
wherein:
X¹ is O or S;
X² is a bond or C₁₋₃alkyl;
P is C₃₋₇cycloalkyl or C₄₋₇cycloalkenyl;
R¹ is hydrogen, C₁₋₆alkyl, cyano, halogen and C₁₋₆alkylhalo, and one or more R¹ may be connected to each other or to one of the atoms that constitutes P to form a bridge or spirocyclo;
R² is hydrogen, C₁₋₃alkyl, fluoromethyl, difluoromethyl, trifluoromethyl, methoxy, fluoromethoxy, difluoromethoxy or trifluoromethoxy;
R⁴ is hydrogen;
Q is a ring containing 4, 5, 6 or 7 atoms independently selected from C, S, O and N, which may be saturated or partially unsaturated and said ring may further contain groups independently selected from SO, SO₂, CO, cyano and CS;
R³ is hydrogen, hydroxy, halogen, nitro, OC₁₋₃alkylhalo, C₁₋₃alkylhalo, C₁₋₃alkyl, C₁₋₃alkoxyC₀₋₃alkyl, C₁₋₃alkanol, cyano, amino or amide;
n is 0, 1, 2, 3 or 4; and
m is 0, 1, 2, 3 or 4;
or N₁-oxides, salts, solvates or solvated salts thereof.

3. The compound according to any one of claims 1 or 2, wherein P is C₃₋₇cycloalkyl substituted with one or more R¹, wherein R¹ is hydrogen, C₁₋₆alkyl, cyano, halogen or C₁₋₆alkylhalo, and one or more R¹ may be connected to each other or to one of the atoms that constitutes P to form a bridge or spirocyclo.

4. The compound according to claim 3, wherein P is C₅₋₇cycloalkyl substituted with one or more R¹, wherein R¹ is methyl.

5. The compound according to any one of claims 1 to 4, wherein X¹ is oxygen.

6. The compound according to any one of claims 1 to 5, wherein X² is a bond.

7. The compound according to any one of claims 1 to 6, wherein R² is hydrogen.

8. The compound according to any one of claims 1 to 7, wherein R⁴ is hydrogen or methyl.

9. The compound according to any one of claims 1 to 8, wherein Q is a ring containing 5, 6 or 7 atoms independently selected from C, O and N, which may be saturated or partially unsaturated.

10. The compound according to any one of claims 1 to 9, wherein R³ is hydrogen, hydroxy, halogen, cyano, C₁₋₃alkyl or C₁₋₃alkoxyC₀₋₃alkyl.

11. The compound according to any one of claims 1 to 10 having a trans-relationship between R¹ and X² on ring P, when P is cyclohexane and R¹ and X² is attached to P at position 4 and 1 respectively.

12. The compounds
N-(trans-4-methylcyclohexyl)-5,6,7,8-tetrahydroquinoxaline-2-carboxamide,
N-(4,4-dimethylcyclohexyl)-5,6,7,8-tetrahydroquinoxaline-2-carboxamide,
or salts, solvates or solvated salts thereof.

13. The compounds
N-(4,4-dimethylcyclohexyl)-3-methyl-5,6,7,8-tetrahydroquinoxaline-2-carboxamide,
8-methyl-N-(trans-4-methylcyclohexyl)-5,6,7,8-tetrahydroquinoxaline-2-carboxamide,
7-hydroxy-5,7-dimethyl-N-(trans-4-methylcyclohexyl)-6,7-dihydro-5H-cyclopenta[b]pyrazine-2-carboxamide,
N-(trans-4-methylcyclohexyl)-6,7,8,9-tetrahydro-5H-cyclohepta[b]pyrazine-2-carboxamide,
7-methyl-N-(trans-4-methylcyclohexyl)-5,6,7,8-tetrahydroquinoxaline-2-carboxamide,
6-methyl-N-(trans-4-methylcyclohexyl)-5,6,7,8-tetrahydroquinoxaline-2-carboxamide,
N-(trans-4-methylcyclohexyl)-6,7-dihydro-5H-cyclopenta[b]pyrazine-2-carboxamide,
N-(trans-4-methylcyclohexyl)-7,8-dihydro-5H-pyrano[3,4-b]pyrazine-2-carboxamide,
N-(trans-4-methylcyclohexyl)-7,8-dihydro-5H-pyrano[3,4-b]pyrazine-3-carboxamide,
7-hydroxy-N-(trans-4-methylcyclohexyl)-5,6,7,8-tetrahydroquinoxaline-2-carboxamide,
6-hydroxy-N-(trans-4-methylcyclohexyl)-5,6,7,8-tetrahydroquinoxaline-2-carboxamide,
N-(4,4-dimethylcyclohexyl)-5,6,7,8-tetrahydroquinoxaline-2-carboxamide 4-oxide and
6,7-dimethyl-N-(4-methylcyclohexyl)-6,7-dihydro-5H-cyclopenta[b]pyrazine-2-carboxamide,
or salts, solvates or solvated salts thereof.

14. A pharmaceutical composition comprising as active ingredient a therapeutically effective amount of the compound according to any one of claims 1 to 13, in association with one or more pharmaceutically acceptable diluent, excipients and/or inert carrier.

15. The pharmaceutical composition according to claim 14, for use in the treatment of Group I mGluR mediated disorders.

16. The compound according to any one of claims 1 to 13, for use in therapy.

17. The compound according to any one of claims 1 to 13, for use in treatment of Group I. mGluR mediated disorders.

18. Use of the compound according to any one of claims 1 to 13, in the manufacture of a medicament for the treatment of Group I mGluR mediated disorders.

19. A method of treatment of Group I mGluR mediated disorders, comprising administrering to a mammal, including man in need of such treatment, a therapeutically effective amount of the compound according to any one of claims 1 to 13.

20. The method according to claim 19, for use in treatment of neurological disorders.

21. The method according to claim 19, for use in treatment of psychiatric disorders.

22. The method according to claim 19, for use in treatment of chronic and acute pain disorders.

23. The method according to claim 19, for use in treatment of gastrointestinal disorders.

24. A method for inhibiting activation of Group I mGluR receptors, comprising treating a cell containing said receptor with an effective amount of the compound according to claim 1 or 2.

25. Processes for the preparation of the compound according to claim 1 or 2, wherein P, Q, X¹, X², R¹, R², R³, R⁴, m and n are, unless otherwise specified, defined as in formula I, comprising of: 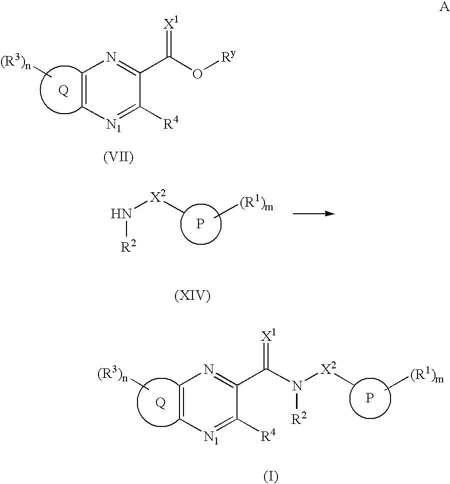
reacting a compound of formula VII, wherein R^{y} is H, with an activating agent followed by the treatment of the resulting acid halide, or otherwise to nucleophiles activated acid derivative, with an amine of formula XIV, to obtain the compound of formula I,
alternatively, 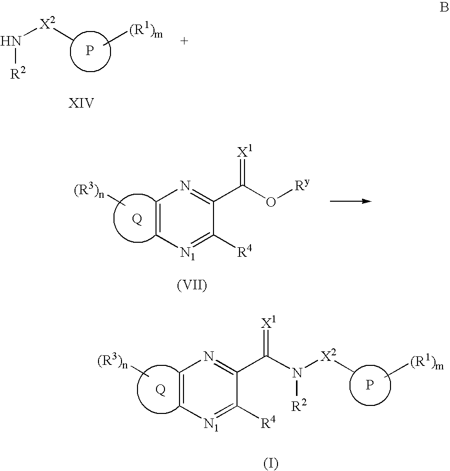
reacting an amine of formula XIV with the compound of formula VII, wherein R^{y} is H, to obtain the compound of formula I, or 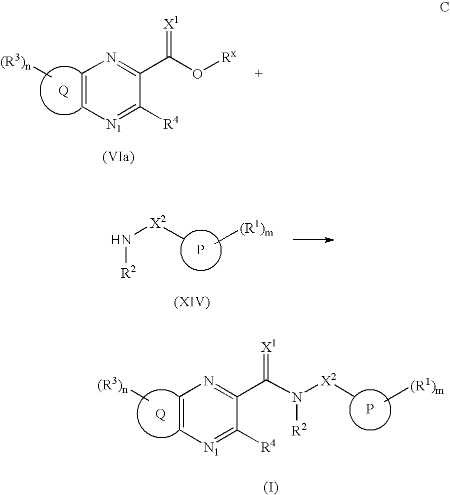
reacting a compound of formula VIa or the N₁-oxide thereof, wherein R^{x} is C₁₋₆ alkyl, with the appropriate amine such as the compound of formula XIV, to obtain the compound of formula I,
or, 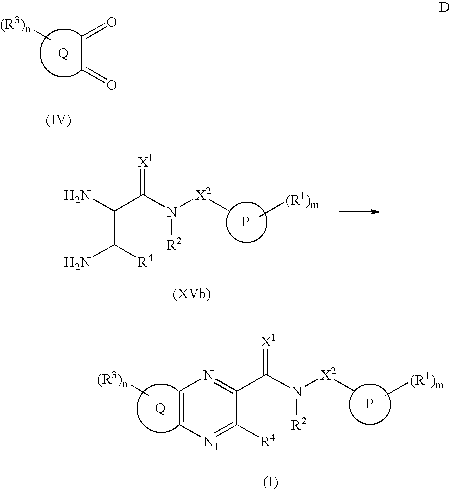
direct condensation of intermediates of formula IV and XVb, to obtain the compound of formula I.

26. Compounds
5,6,7,8-tetrahydro-quinoxaline-2-carboxylic acid methyl ester and
5,6,7,8-tetrahydro-quinoxaline-2-carboxylic acid.

27. Compounds
3-methyl-5,6,7,8-tetrahydro-quinoxaline-2-carboxylic acid ethyl ester,
3-methyl-5,6,7,8-tetrahydro-quinoxaline-2-carboxylic acid,
2,3-diamino-N-(4-methyl-cyclohexyl)-propionamide,
4-(tert-butyl-diphenyl-silanyloxy)-cyclohexane-1,2-dione,
6,7-dimethyl-6,7-dihydro-5H-cyclopentapyrazine-2-carboxylic acid methyl ester,
5,6,7,8-tetrahydro-quinoxaline-2-carboxylic acid methyl ester and
5,6,7,8-tetrahydro-quinoxaline-2-carboxylic acid.

28. The compounds according to claims 26 and 27, for use as an intermediate in the preparation of the compound according to claim 1.
